(19) United States
(12) Patent Application Publication
(10) Pub. No.: US 2005/0201971 A1
(43) Pub. Date: Sep. 15, 2005

(54) Advertisement method

(76) Inventors: Radhakrishnan Janardanan Nair, Kobe (JP); Ryohei Ohtani, Nishinomiya (JP); Mayumi Ashiya, Kobe (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/073,505

(22) Filed: Mar. 7, 2005

Related U.S. Application Data
(60) Provisional application No. 60/551,514, filed on Mar. 9, 2004.

(51) Int. Cl. G06F 17/60; A61K 9/26

(52) U.S. Cl. 424/76.1; 705/1

(57) ABSTRACT

The present invention is directed to a method for advertising a deodorizer. The method has the steps of using a color indicator and a color change reaction caused by the color indicator; and visualizing the malodor removal performance of the product by the color change. The advertising method also has the steps of preparing a promotion product and a comparative product; preparing at least two separate closed spaces; and then, in any order: placing the promotion product inside one of the closed spaces; placing a material inside each closed space, wherein the material comprises a color indicator; adding a malodor substance to each of the closed spaces to thereby cause a color change to the color indicator. Finally, the last step is that of showing the difference of the material color between the promotion product and the comparative product. These steps are visually recorded to form an advertisement and wherein the advertisement is transferred electronically.

### CROSS REFERENCE TO RELATED APPLICATION

This application claims the benefit of U.S. Provisional Application No. 60/551,514, filed Mar. 9, 2004.

### FIELD OF THE INVENTION

The present invention relates to an advertising method. Specifically, the present invention relates to an advertising method for deodorant.

### BACKGROUND OF THE INVENTION

There are many types of malodors in society. Especially, there are many uncomfortable malodors at home. For example, when foods are rotten, amine type or hydrogen sulfide type malodors may cause kitchens or refrigerators to smell unpleasant. Also, ammonia type or mercaptan type malodors may make toilets smell unpleasant. In order to deodorize or remove these malodors, many types of deodorizers have been developed and sold in the market.

However, as there are many deodorizers in the market, it is difficult for consumers to know which product is best for them. In fact, it is not easy to show the performance of the deodorizer in advertisements as these advertisement have difficulties to show malodor removal performance efficiently. For example, consumers may not be able to know deodorant performance sufficiently via a television commercial or other advertisement.

Therefore, there is a need to provide a method for advertising deodorizer products which shows their malodor removal performance.

### SUMMARY OF THE INVENTION

The present invention relates to a method for advertising a deodorizer. The method includes the steps of using a color indicator and a color change reaction caused by the color indicator; and visualizing the malodor removal performance of the product by the color change.

In another embodiment, the present invention relates to a method for advertising deodorizers. The method has steps of preparing a promotion product and a comparative product, and then, in any order: placing these two products inside two separate closed spaces, placing a material containing a color indicator inside each closed space, and adding a malodor substance that causes a color change to the color indicator. Finally, showing the difference of the material color between the promotion product and the comparative product. These steps are visually recorded to form an advertisement and the advertisement is transferred electronically.

As malodors are generally invisible, it is difficult to show the malodor removal performance of a deodorizer to consumers on TV or internet. The present invention provides a method for advertising deodorizers effectively by visualizing the malodor removal performance of the deodorizer. As a result, such a visual advertisement is highly convincing to show consumers the effectiveness of the deodorizer, and the advertisement may cause consumers to increase their motivation to purchase the promotion product with high malodor removal performance. In addition, such a method may be useful to measure the malodor removal efficacy of various deodorizers, which up until now has been difficult for consumers to visibly quantify.

### DETAILED DESCRIPTION OF THE INVENTION

Definitions

All percentages, ratios and proportions herein are by weight of the composition, unless otherwise specified. All temperatures are in degrees Celsius (° C.) unless otherwise specified.

As used herein, the term “comprising” and its derivatives are intended to be open ended terms that specify the presence of the stated features, elements, components, groups, integers, and/or steps, but do not exclude the presence of other, unstated features, elements, components, groups, integers, and/or steps. This definition also applies to words of similar meaning, for example, the term “have”, “include”, “be provided with” and their derivatives. This term encompasses the terms “consisting of” and “consisting essentially of”.

As used herein, the term “deodorizer” means a product which can remove malodors from air such as living room, toilet, kitchen. The deodorizer of the present invention includes placement type deodorizers. Also, the deodorizer of the present invention includes both deodorizers working by electricity and working without electricity.

As used here, the term “closed space” means a substantially air-tight volume of space which the deodorizer, and material are placed.

Advertisement Method of the Present Invention

The method of the present invention is intended to advertise one or more deodorizers by using a color indicator and a color change reaction between the color indicator and the deodorizer and visualizing the malodor removal performance of the product by the color change.

The color indicator of the present invention can be selected any material depending on what types of deodorizers are advertised. Preferably, the color indicator is a pH indicator because many malodors may belong to either acidic or base type malodor. More preferably, the pH indicator is selected from the group consisting of methyl red, methyl orange, methyl yellow, thymol blue, thymolphtalein, bromophenol blue, phenolphthalein, Litmus paper and a mixture thereof.

More specifically, the method of the present invention may preferably comprise the following steps.

As a first step, a deodorizer product which is going to be advertised and/or promoted (hereinafter, called “promoted product”) is prepared. One separate closed space is also prepared. Then, the promoted product is placed inside of the closed spaces. The volume of the closed space can be decided depending on the size of the deodorizers or concentration of a gas, but plastic bag or room is preferably used. The closed space is preferably formed of a clear or translucent material, so that any color changes are able to be easily viewed from outside of the closed spaces. If there are more than two products such as comparative product, then a closed space is separately prepared for each product. Then, a material is placed inside of the space. The material has a color indicator. If not, it is also possible to put the color indicator to the promoted product. Any material can be used as long as the material can absorb, hold and/or be impregnated with the color indicator. Preferable materials include paper, a fabric, a non-woven material and/or plastic. A laminate sheet is also preferable having two layers: one layer being a water-absorbing layer and the other layer being a water-resistant layer. Thus, if using laminate board, it is possible to add a color indicator solution to the water-absorbing layer but prevent leakage of the color indicator solution. Also, the material can be any form, but a sheet form is preferable. In addition, the color of the material is preferably white because the color change is clearly visible for consumers.

Then, a malodor substance is added to the space by any means such as a pump, syringe, etc. as long as the space is air-tight except the malodor substance. Any malodor substance can be used as long as the gas can interact with the color indicator and cause a color change. Preferable malodor substance is selected from the group consisting of ammonia, trimethyalmine, acetic acid, isovaleric acid and a mixture thereof, which are representative of undesirable odors encountered by consumers of deodorizer products. The color change interaction is preferably reversible so that it is possible to know the change of the gas concentration in the space after the deodorizer starts to absorb and/or neutralize the malodor substance. Also, a vivid color change such as from red or orange to transparent is preferable to show the malodor removal performance remarkably to consumers. From that standpoint, the combination of ammonia with phenolphthalein or other alkaline pH indicator is preferable.

Finally, the color change difference of the material between the promotion product and the comparative product is shown. It is better to show the comparison immediately after the malodor substance is added and for a couple of minutes thereafter. As long as the promotion product has better and faster malodor removal performance, the color change may noticeably occur compared with comparative product (when using comparative product).

These steps are then visually recorded to form an advertisement and then transferred electronically or manually. For example, the advertisement may be transferred by an electronic cable as an advertisement of deodorizers, broadcast for television, made available on the internet, provided on a videotape or other electronic media, transferred via radio waves, telephone signals and/or telephone lines, delivered printed materials such as magazines, newpapers, product pamphlets etc. The term “broadcast for television” specifically includes transfer via cable television signals and via internet cable.

### EXAMPLE 1

Two transparent plastic bags (size: 5 liters) representing closed spaces, with a sealable tube and two white sheets, representing a material, are prepared. Each white sheet is a white thermo bonded laminate (one side is a nonwoven material, the other side is plastic film). The size of the white sheet is 23 cm×17 cm. The nonwoven side of the white sheet contains include 2.2 milliliters (ml) of phenolphthalein solution. The phenolphthalein solution is made by dissolving 100 mg phenolphthalein in 125 ml ethanol-water mixture (25 ml ethanol and 100 ml deionized water). Each white sheet is inserted into a separate bag, and each bag is fixed by tape onto a white colored wall. Two Petri dishes (4.5 cm diameters each) are prepared and 15 g of a promotion product is placed on the first Petri dish, and 15 g comparative product is placed on the other Petri dish. One Petri dish is inserted into each of the bags. Then, these bags are sealed and 40 ml of ammonia gas is injected by a syringe through the tube of the bag.

Both sheets inside the bags start to turn red and a photo of each sheet is taken.

After 15 minutes, the color of the sheet inside the bag having the promotion product starts to turn white. A picture of these bags is also taken. The catch copy for the promotion product is: malodor catcher grasps malodors. In contrast, the color of the sheet inside the bag having the comparative product is still red. The catch copy for the comparative product is: malodor remains.

After 30 minutes, another picture of these plastic bags is taken. The color of the sheet inside the bag having the promotion product has become almost white. The catch copy for the promotion product is: malodor is almost gone. In contrast, the color of the sheet inside the bag having the comparative product is still red. The catch copy for the comparative product is: malodor still remains.

### EXAMPLE 2

An advertising method as described in Example 1 is prepared, except that the sheet is a piece of absorbent paper, the entire method is videotaped and sent via a coaxial cable to a television screen where it is viewed. In addition, a clock indicating the elapsed time is affixed to the wall near the bags.

### EXAMPLE 3

An advertising method as described in Example 2 is prepared, except that the entire method is videotaped and then posted on a web page on a local area computer network where it is available for downloading onto individual computers within the secure system.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for advertising a deodorizer comprising the steps of using a color indicator and a color change reaction caused by the color indicator; and visualizing the malodor removal performance of the product by the color change.

2. A method for advertising a deodorizer comprising the steps of:
(a) preparing a promotion product;
(b) preparing a closed space;
(c) placing the promotion product inside one of the closed spaces;
(d) placing a material inside the closed space, wherein the material comprises a color indicator;
(e) adding a malodor substance to each of the closed spaces to thereby cause a color change to the color indicator; and
(f) showing the difference of the material color between the promotion product and the comparative product,
wherein steps (c) to (e) may be conducted in any order, wherein these steps are visually recorded to form an advertisement, and wherein the advertisement is transferred electronically.

3. The method according to claim 2, wherein the material is selected from the group consisting of paper, a fabric, a non-woven material, a plastic and a mixture thereof.

4. The method according to claim 2, wherein the color indicator is a pH indicator.

5. The method according to claim 2, wherein the malodor substance is selected from the group consisting of ammonia, trimethyl amine, isovaleric acid, acetic acid and a mixture thereof.

6. The method according to claim 2, wherein the advertisement is transferred to a destination selected from the group consisting of a television, a computer, an internet, a paper magazine, a paper newspaper, a paper pamphlet and a mixture thereof.

7. A method for advertising deodorizers comprising the steps of:
(a) preparing a promotion product and a comparative product;
(b) preparing two separate closed spaces;
(c) placing the promotion product inside one of the closed spaces;
(d) placing the comparative product inside the other of the closed spaces;
(e) placing one piece of white paper inside each closed space, wherein the paper comprises a pH indicator;
(f) adding a malodor substance to each of the closed spaces to thereby cause a color change to the pH indicator; and
(g) showing the difference of each paper color between the promotion product and the comparative product,
wherein steps (c) to (f) may be conducted in any order, wherein these steps visually recorded to form an advertisement, and wherein the advertisement is transferred electronically.

8. The method according to claim 7, wherein the pH indicator is phenolphthalein and the gas is ammonia gas.
